(19) United States
(12) Patent Application Publication
(10) Pub. No.: US 2023/0266346 A1
(43) Pub. Date: Aug. 24, 2023

(54) METHODS FOR QUANTIFYING POTENCY OF REGENERATIVE IMMUNOTHERAPIES

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Timothy WARBINGTON, Phoenix, AZ (US)

(73) Assignee: CREATIVE MEDICAL TECHNOLOGIES, INC., Phoenix, AZ (US)

(21) Appl. No.: 18/173,052

(22) Filed: Feb. 22, 2023

Related U.S. Application Data
(60) Provisional application No. 63/313,313, filed on Feb. 24, 2022.

(51) Int. Cl. G01N 33/74; G01N 33/68

(52) U.S. Cl. CPC G01N 33/74; G01N 33/6863; G01N 2333/475; G01N 2333/52; G01N 2800/52

(57) ABSTRACT

Disclosed are means, methods and compositions of matter useful for quantifying the potency of regenerative therapeutics based on utilization of immunotherapies to induce tissue repair. In one embodiment said immunotherapy with regenerative activity is a T regulatory cell based therapy for stroke whose potency is quantified by assessment of one or more from the following: a) basal production of regenerative factors; b) induced production of regenerative factors; c) ability to prevent apoptosis of a target cell of interest; d) ability to stimulation proliferation of a target cell; and e) ability to induce proliferation of a progenitor cell belonging to tissue type of which therapy is desired.

### CROSS-REFERENCE TO RELATED APPLICATIONS

This application claims priority to U.S. Provisional Application No. 63/313,313, filed Feb. 24, 2022, titled “Methods for Quantifying Potency of Regenerative Immunotherapies”, which is hereby incorporated by reference in its entirety.

### FIELD OF THE INVENTION

The teachings herein are directed to methods and compositions of matter useful for quantifying the potency of regenerative therapeutics based on utilization of immunotherapies to induce tissue repair.

### BACKGROUND

For approval of therapeutics by regulatory agencies, there must be ways to ensure that batch to batch consistency exists. Potency assays are intended to predict the therapeutic activity of a cellular composition by detecting or measuring one or more biological markers that are linked to one or more physiological properties. Because the therapeutic mechanisms of action can vary between therapeutic cellular compositions, however, it can be difficult to identify a biological function that is reproducibly linked to the relevant biological or biophysical properties. In addition, because the therapeutic mechanisms of action can rely on complex biological pathways, it can be especially challenging to identify which product attributes are most relevant to determining potency. Nonetheless, it is extremely important to develop potency assays that reflect the cellular compositions therapeutic properties and provide a reliable measure of production batch-to-batch consistency.

In the area of regenerative medicine, numerous organs have been shown to possess their own stem cell compartments which become activated during tissue injury. These stem cells, or sometimes referred to has endogenous progenitor cells, have a role in repair, and in some studies it has been shown that the higher number of these cells that are found in the injured organ, the more likely recovery is possible. One example of these endogenous progenitor cells is in the brain. It has been shown that cerebral neurogenic niches such as in the subventricular zone are activated in response to brain injury such as trauma [1-12], or stroke [1, 13-23]. In the example of stroke, administration of agents that stimulate neurogenesis such as docosonoids, [24], angiogenin [25], GDNF [26], BDNF [27], BMP-7 [28], pifithrin-alpha [29], erythropoietin [30], estradiol [31], progranulin [32], retinoic acid [33], GADD45b[34], and hCG [12, 35]

The invention provides means of quantifying the potency of regenerative medicine therapies that are based on immunotherapy through providing a series of tests for assessment of mitogenic activity of the regenerative immunotherapy cells, as well as ability to protect certain tissues from apoptosis.

### SUMMARY

Preferred embodiments include methods of quantifying potency of an immunologically based regenerative medicine therapy in which said potency is assessed by one or more of the following methods: a) quantification of basal growth factor production; b) quantification of induced growth factor production; c) quantification of basal immune regulatory factor production; d) quantification of induced growth factor production; e) assessment of ability of basal conditioned media from said immunologically based regenerative medicine therapy to reduce apoptosis in cells representing cells of the target tissue to be treated; f) assessment of ability of basal conditioned media from said immunologically based regenerative medicine therapy to induce regeneration in cells representing cells of the target tissue to be treated; g) assessment of ability of induced conditioned media from said immunologically based regenerative medicine therapy to reduce apoptosis in cells representing cells of the target tissue to be treated; and h) assessment of ability of induced conditioned media from said immunologically based regenerative medicine therapy to induce regeneration in cells representing cells of the target tissue to be treated.

Preferred methods include embodiments wherein said immunologically based immunotherapy comprises an immune cell that has been reprogrammed by a regenerative cell.

Preferred methods include embodiments wherein said reprogramming endows said immune cell with ability to possess regenerative properties.

Preferred methods include embodiments wherein said regenerative properties are angiogenic in nature.

Preferred methods include embodiments wherein said regenerative properties are neurogenic in nature.

Preferred methods include embodiments wherein said regenerative properties are antiapoptotic in nature.

Preferred methods include embodiments wherein said regenerative properties are mitogenic in nature.

Preferred methods include embodiments wherein said regenerative properties are antiinflammatory in nature.

Preferred methods include embodiments wherein potency of cells is assessed by quantification of ability to stimulate angiogenesis in an in vitro assay.

Preferred methods include embodiments wherein said in vitro assay is stimulation of proliferation of human umbilical vein endothelial cells.

Preferred methods include embodiments wherein said in vitro assay is stimulation of migration of human umbilical vein endothelial cells.

Preferred methods include embodiments wherein said in vitro assay is stimulation of activation of human umbilical vein endothelial cells.

Preferred methods include embodiments wherein said in vitro assay is assessment of angiogenic cytokines produced by cells being assayed.

Preferred methods include embodiments wherein said angiogenic cytokines are selected from a group comprising of: G-CSF, GM-CSF, 1-309, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, FGF-1, FGF-2, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Ax1, BTC, CCL28, CTACK, CXCL16, ENA-78, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, and Galectin-3.

Preferred methods include embodiments wherein said cytokines produced by said cells being assayed are produced at a basal level.

Preferred methods include embodiments wherein said cytokines produced by said cells being assayed are produced after stimulation of said cells.

Preferred methods include embodiments wherein said cells are stimulated by one or more means selected from a group comprising of: a) hypoxia; b) hypertonic stress; c) hypotonic stress; d) hyperthermia; e) stimulation with a mitogen; f) stimulation with a cytokine; and g) stimulation with a toll like receptor agonist.

Preferred methods include embodiments wherein neurogenic potential is assessed by ability to stimulate proliferation of neuronal progenitors in vitro.

Preferred methods include embodiments wherein said neuronal progenitors express CD133.

Preferred methods include embodiments wherein said neuronal progenitors express CD105

Preferred methods include embodiments wherein said neuronal progenitors express c-kit.

Preferred methods include embodiments wherein said neuronal progenitors express tie-1.

Preferred methods include embodiments wherein said neuronal progenitors express Mushashi.

Preferred methods include embodiments wherein said neuronal progenitors express nestin.

Preferred methods include embodiments wherein said neuronal progenitors express SOX2.

Preferred methods include embodiments wherein said neuronal progenitors express OCT-4.

Preferred methods include embodiments wherein said neuronal progenitors express Nanog.

Preferred methods include embodiments wherein said neuronal progenitors express KLF-4.

Preferred methods include embodiments wherein said neuronal progenitors express FGF-1 receptor.

Preferred methods include embodiments wherein said neuronal progenitors express c-Met.

Preferred methods include embodiments wherein said neuronal progenitors express FGF-2 receptor.

Preferred methods include embodiments wherein said neuronal progenitors express interferon gamma receptor.

Preferred methods include embodiments wherein HGF-1 is used as a method of assessing potency of said regenerative immunotherapy population.

Preferred methods include embodiments wherein HGF-1 assessed is assessed subsequent to culture of said regenerative immunotherapy population with anti-CD3 and anti-CD28 antibodies bound to beads.

Preferred methods include embodiments wherein said beads are DynaBeads.

Preferred methods include embodiments wherein regenerative immunotherapy cells are released for use if production of HGF-1 is above 30 nanograms per 1 million regenerative immunotherapy cells stimulated with 10 million DynaBeads for a period of 24 hours.

### DETAILED DESCRIPTION OF THE INVENTION

In one embodiment of the invention, ImmCelz potency is assessed by quantification of growth factor production after stimulation of ImmCelz with phytohmagluttinin (PHA). In one embodiment of the invention ImmCelz is generated by culture of patient peripheral blood mononuclear cells (PBMC) with conditioned media from activated perinatal stem cells, such as umbilical cord cells. In one embodiment of the invention the practitioner of the invention esuspends ImmnCelz cells at 2e6 cells/mL in complete medium (e.g. IMDM+2 mM L-Glutamine+10% FBS). The practitioner of the invention then adds PHA to a final concentration of 5-10 ug/mL. Incubate for 5 days in an appropriate vessel for your culture size (both flasks and plates will work, even if the latter is not TC coated). 2 Incubate for 5 days at 37 C. 3 On day 5-6, growing clusters and acidified media should be visible by naked eye. Count cell density. If density is over 1e6 cells/mL, adjust to 1e6 cell/mL with media. If density is at or below 1e6 cells/mL, spin down half of the culture and resuspend in the same volume with fresh media. Add IL2 to a final concentration of 50 U/mL. Incubate for 2-3 days. 4 After 2-3 days, and every 2-3 days thereafter, add IL2 to a final concentration of 50 U/mL (always assume all of the IL2 from previous feedings is completely consumed). If cell numbers drop, increase IL2 dose by 50 U/mL. Use this increased dose for subsequent feedings. Around day 10-14, restimulate cells with 5-10 ug/mL PHA if you will not be freezing them. 5 By day 10, I usually observe that CD4 T cells preferentially grow compared to CD8 T cells (CD4:CD8 of >70:30), and such CD4 T cells express moderate to high levels of HLA class I, making them ideal for assay of class I-restricted effector cells.

Provided herein are methods for assessing cell potency, e.g., assessing the potency of cells in a preparation of cells (e.g., a preparation of cells that belongs to a lot of cells intended for therapeutic use). The methods provided herein utilize an increased cell culture time, e.g., recovery time, prior to assessing cell potency, as compared to standard cell potency assays used in the art. It has been determined that incorporating such an increased cell culture time results in increased assay reliability and decreased variability across cell preparations taken from same cell lot, as well as cell preparations comprising the same cell type but taken from different cell lots.

In a specific embodiment, provided herein is a method for assessing cell potency of one or more cells used in regenerative immunotherapy, said method comprising the following steps (i) culturing a population of cells, e.g., a population of cells intended/suitable for use as a cell therapy, for a time period that minimizes variability in cell number once the cells have been cultured for the time period; and (ii) performing an assay to determine whether the cells produce a marker that acts as a surrogate for potency of the cells. The assay may involve assessment of functional activities of the cell itself or it may involve quantification of gene or protein expression. In another specific embodiment, provided herein is a method for assessing cell potency, said method comprising the following steps (i) obtaining a population of cells, e.g., a population of cells intended/suitable for use as a cell therapy; (ii) culturing the cells for a time period that minimizes variability in cell number once the cells have been cultured for the time period; and (iii) performing an assay to determine whether the cells produce a marker that acts as a surrogate for potency of the cells. In another specific embodiment, provided herein is a method for assessing cell potency, said method comprising the following steps (i) culturing a population of cells, e.g., a population of cells intended/suitable for use as a cell therapy, for about 48 hours; and (ii) performing an assay to determine whether the cells produce a marker that acts as a surrogate for potency of the cells. In another specific embodiment, provided herein is a method for assessing cell potency, said method comprising the following steps (i) obtaining a population of cells, e.g., a population of cells intended/suitable for use as a cell therapy; (ii) culturing the cells for about 48 hours; and (iii) performing an assay to determine whether the cells produce a marker that acts as a surrogate for potency of the cells. Markers associated with regeneration that are soluble in nature can be utilized for assessment of potency of regenerative cells. In one example assessment of the regenerative cytokine HGF is performed. HGF is of interest because it has been shown to possess regenerative activities in numerous situations including cardiac [36-74], hepatic [75-92], neuronal [26, 93-108], and nephrotic [109-138], conditions. HGF is also interesting as a potency marker due to its ability to induce immunological tolerance and in some cases prevent graft rejection [139-141], autoimmune myocarditis [142], multiple organ failure [143-151],

In another specific embodiment, provided herein is a method for assessing cell potency, said method comprising the following steps (i) culturing a population of cells, e.g., a population of cells intended/suitable for use as a cell therapy, for about 48 hours; and (ii) performing an assay to determine whether the cells produce interleukin-10 (IL-10), wherein expression of IL-10 is a surrogate for potency of the cells. In some embodiments said cells are cultured with one or more activating agents. In one embodiment said activating agent is antibodies to receptors found on immune cells whose potency is being assessed. If the immune cell is a B cell, said antibodies are antibodies activatory to B cells. Antibodies known to be activitory to B cells including antibodies to CD19, antibodies to CD20 and antibodies to B cell receptor. If the immune cell is a T cell, said antibodies are antibodies activatory to T cells. Antibodies known to be activitory to B cells including antibodies to CD3, antibodies to CD5 and antibodies to T cell receptor. In some cases activation is performed by exposure to a lectin. Lectins useful for practice of the invention including phytohemagluttinin, conconavalin A, pokeweed mitogen and lipopolysaccharide.

In another specific embodiment, provided herein is a method for assessing cell potency, said method comprising the following steps (i) obtaining a population of cells, e.g., a population of cells intended/suitable for use as a cell therapy; (ii) culturing the cells for about 48 hours; and (iii) performing an assay to determine whether the cells produce VEGF wherein expression of VEGF is a surrogate for potency of the cells.

In another specific embodiment, provided herein is a method for assessing cell potency, said method comprising the following steps (i) culturing a population of cells, e.g., a population of cells intended/suitable for use as a cell therapy, for a time period that minimizes variability in cell number once the cells have been cultured for the time period; (ii) further culturing the cells in a medium that comprises one or more agents capable of inducing production of one or more genes in the cells, e.g., to cause increase production of one or more proteins by the cells; and (iii) performing an assay to determine whether the cells produce a marker that acts as a surrogate for potency of the cells. Genes associated with potency of said cells include: interleukin 1, interleukin 2, interleukin 3, interleukin 4, interleukin 7, interleukin 10, interleukin 13, interleukin 20, interleukin 35, TGF-beta, IGF, EGF, FGF-1, FGF-2, FGF-5, angiopoietin, leukemia inhibitory factor, and PDGF.

In another specific embodiment, provided herein is a method for assessing cell potency, said method comprising the following steps (i) obtaining a population of cells, e.g., a population of cells intended/suitable for use as a cell therapy; (ii) culturing the cells for a time period that minimizes variability in cell number once the cells have been cultured for the time period; (iii) further culturing the cells in a medium that comprises one or more agents capable of inducing production of one or more genes in the cells, e.g., to cause increase production of one or more proteins by the cells; and (iv) performing an assay to determine whether the cells produce a marker that acts as a surrogate for potency of the cells.

In another specific embodiment, provided herein is a method for assessing cell potency, said method comprising the following steps (i) culturing obtaining a population of cells, e.g., a population of cells intended/suitable for use as a cell therapy, for about 48 hours; (ii) further culturing the cells in a medium that comprises one or more agents capable of inducing production of one or more genes in the cells, e.g., to cause increase production of one or more proteins by the cells; and (iii) performing an assay to determine whether the cells produce a marker that acts as a surrogate for potency of the cells. In another specific embodiment, provided herein is a method for assessing cell potency, said method comprising the following steps (i) obtaining a population of cells, e.g., a population of cells intended/suitable for use as a cell therapy; (ii) culturing the cells for about 48 hours; (iii) further culturing the cells in a medium that comprises one or more agents capable of inducing production of one or more genes in the cells, e.g., to cause increase production of one or more proteins by the cells; and (iv) performing an assay to determine whether the cells produce a marker that acts as a surrogate for potency of the cells.] In another specific embodiment, provided herein is a method for assessing cell potency, said method comprising the following steps (i) culturing a population of cells, e.g., a population of cells intended/suitable for use as a cell therapy, for about 48 hours; (ii) further culturing the cells in a medium that comprises an agent capable of inducing production of HGF by the cells; and (iii) performing an assay to determine whether the cells produce HGF, wherein expression of HGF is a surrogate for potency of the cells. In a specific embodiment, said agent capable of inducing production of HGF by the cells is anti-CD3 antibody. In some cases, stimulation with anti-CD3 may be combined with stimulation with anti-CD28. In another specific embodiment, provided herein is a method for assessing cell potency, said method comprising the following steps (i) obtaining a population of cells, e.g., a population of cells intended/suitable for use as a cell therapy; (ii) culturing the cells for about 72 hours; (iii) further culturing the cells in a medium that comprises an agent capable of inducing production of HGF by the cells; and (iv) performing an assay to determine whether the cells produce HGF, wherein expression of HGF is a surrogate for potency of the cells. In a specific embodiment, said agent capable of inducing production of HGF by the cells is anti-CD3 antibody. In some cases, stimulation with anti-CD3 may be combined with stimulation with anti-CD28.

The methods for assessing for cell potency described herein can be carried using any immunological cell type, e.g., any cell type intended/suitable for use as a therapeutic. In preferred embodiments the assays are utilized for assessment of potency of cells used for regenerative immunotherapy. Exemplary cell types that can be used in the methods described herein include, without limitation, immune cells that have been exposed to regenerative cells. Regenerative cells useful for the endowment of regenerative properties to immune cells include but are not limited to mesenchymal stem cells, bone marrow-derived mesenchymal stem cells (BM-MSCs), tissue culture plastic-adherent CD34−, CD10+, CD105+, CD200+ placental stem cells, embryonic stem cells, embryonic germ cells, induced pluripotent stem cells, mesenchymal stem cells, bone marrow-derived mesenchymal stem cells, bone marrow-derived mesenchymal stromal cells, tissue plastic-adherent placental stem cells (PDACs), umbilical cord stem cells, amniotic fluid stem cells, osteogenic placental adherent cells (OPACs), adipose stem cells, limbal stem cells, dental pulp stem cells, myoblasts, endothelial progenitor cells, neuronal stem cells, exfoliated teeth derived stem cells, hair follicle stem cells, dermal stem cells, parthenogenically derived stem cells, reprogrammed stem cells, amnion derived adherent cells [152], and side population stem cells [153-159]. In certain embodiments, the cells used in the methods provided herein are from a lot of cells that has been previously prepared. In certain embodiments, when the cells used in the methods provided herein are from a lot of cells that has been previously prepared, said cells have been preserved, e.g., cryopreserved, prior to use in the method. In certain embodiments, when the cells used in the methods provided herein are from a lot of cells that has been previously prepared, said cells have been not been preserved, e.g., have not been cryopreserved, prior to use in the method. In a specific embodiment, the time period used in the methods provided herein that minimizes variability in cell number is greater than 24 hours. In another specific embodiment, the time period used in the methods provided herein that minimizes variability in cell number is or is about 48 hours. In another specific embodiment, the time period used in the methods provided herein that minimizes variability in cell number is or is about 65 hours. In certain embodiments, the time period used in the methods provided herein that minimizes variability in cell number is about 30 hours, about 36 hours, about 42 hours, about 48 hours, about 54 hours, about 60 hours, or about 66 hours. In certain embodiments, the time period used in the methods provided herein that minimizes variability in cell number is between 30 hours to 36 hours, 36 hours to 42 hours, 42 hours to 48 hours, 48 hours to 54 hours, 54 hours to 60 hours, or 60 hours to 66 hours.

In certain embodiments, the marker that acts as a surrogate for potency of the cells in the population of cells correlates with the mechanism of action (MOA) of the cells for which potency is being assessed using a method described herein. In certain embodiments, the marker that acts as a surrogate for potency of the cells in the population of cells correlates with immunosuppressive activity of the cells. In a specific embodiment, expression of PGE2 is a surrogate for potency of the cells used in the methods described herein. In another specific embodiment expression of one of one more of the following proteins (and/or the genes that encode them) is/are a surrogate for potency of the cells used in the methods described herein: BLC, Eotaxin-1, Eotaxin-2, G-CSF, GM-CSF, 1-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Ax1, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163 , Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3 , LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. ANG, EGF, ENA-78, FGF2, Follistatin, G-CSF, GRO, HGF, IL-6, IL-8, Leptin, MCP-1, MCP-3, PDGFB, PLGF, Rantes, TGFBI, Thrombopoietin, TIMPI, TIMP2, uPAR, VEGF, VEGFD, angiopoietin-1, angiopoietin-2, PECA M-1 (CD3 1; platelet endothelial cell adhesion molecule), laminin and/or fibronectin. In a specific embodiment, said cells are PBMC that have been exposed to factors generated by stem cells. In another specific embodiments said PBMC have been exposed to stem cells that have been previously activated. In another specific embodiments said PBMC have been exposed to mesenchymal stem cells that have been previously activated.

Detection of a marker that acts as a surrogate for cell potency can be accomplished using any method known in the art. In a specific embodiment, markers that act as a surrogate for cell potency are detected by ELISA. In another specific embodiment, markers that act as a surrogate for cell potency are detected by use of a MultiplexBead Assay. In another specific embodiment, markers that act as a surrogate for cell potency are detected using an assay that measures gene expression, e.g., RT-PCR. In certain embodiments, the method of detection of a surrogate for cell potency requires collection of the conditioned media from the cell culture, e.g., collection of the medium from the cell culture after the recovery time and/or collection of the medium from the cell culture after further culturing the cells in a medium that comprises one or more agents capable of inducing production of one or more genes in the cells. In certain embodiments, the cells used in the methods described herein are adherent cells. Culturing of such cells in the methods described herein can be accomplished using, e.g., tissue culture plates, e.g., 6-, 24-, 48-, or 96-well tissue culture plates. In a specific embodiment, 48-well tissue culture plates are used to culture adherent cells in the methods described herein. In certain embodiments, the cells used in the methods described herein are non-adherent cells. Culturing of such cells in the methods described herein can be accomplished, e.g., using tissue culture plates, e.g., 6-, 24-, 48-, or 96-well tissue culture plates, or by using tubes, flasks, and/or other containers known in the art for culturing cells in suspension.

In some embodiments of the invention, potency of cells used for “regenerative immunotherapy” is assessed by their ability to suppress inflammation. It is known that inflammation is associated with production of various cytokines which mediate the biological characteristics that are representative of inflamed tissue such as neutrophil accumulation, enhanced macrophage activity, and vasodilation. One of the prototypic inflammatory cytokines is tumor necrosis factor (TNF)-alpha. This molecule is cause of pathology in numerous inflammatory diseases ranging from rheumatoid arthritis [160], to inflammatory bowel disease [161], to depression [162-164]. Additionally, a numerous types of acute inflammation such as bone fractures [165-167], burns [168-170], and heart attack [171-173], are also associated with release of TNF-alpha. TNF-alpha is considered a mediator of the “innate” immune response. One would expect that treating cells with this inflammatory cytokine would induce a “priming” effect, similar to what hypoxia induces. Indeed, pretreatment with TNF-alpha has been shown to induce expression of growth factors such as VEGF [174], HGF [175], IGF-I [176, 177]. Other studies have shown that TNF-alpha upregulation production of immune modulators such as PGE-2 [178] and also markedly augments ability of cells to stimulate angiogenesis [179].

Adaptive immune response cells, such as T cells, also produce cytokines which modulate MSC activity. During inflammatory responses, T cells produce the cytokine interferon gamma, which is known to cause activation of various innate immune cells, thus helping pathogen clearing. It appears that MSC serve a feedback inhibitory role to inflammatory cytokines by become activated. Perhaps this is because the physiological role of MSC is to heal tissue that has been damaged, and many times, tissue is damaged as collateral damage after an immune response. Interferon gamma has been shown to affect MSC in the following ways: a) increase expression of the immune suppressive enzyme indolamine 2,3 deoxygenase [180, 181]; b) enhance ability of MSC to treat stroke [182]; c) increases production of immune suppressive exosomes [183-185]; d) reduces MSC apoptosis [186]; and e) enhances ability of MSC to stimulate production of T regulatory cells [187].

In certain embodiments, determination of a time period that minimizes variability in cell number once the cells have been cultured for the time period is accomplished quantitatively. In a specific embodiment, determination of a time period that minimizes variability in cell number once the cells have been cultured for the time period is accomplished by counting the cells in the cell culture at a given time point and comparing the number of cells counted to the number of cells counted from a culture of the same cells at a different, e.g., earlier or later, time point. In another specific embodiment, determination of a time period that minimizes variability in cell number once the cells have been cultured for the time period is accomplished by visually inspecting the cells in the cell culture at a given time point, determining the percent confluence of the cell culture, and comparing the percent confluence of the cell culture to the percent confluence of cells visually inspected from a culture of the same cells at a different, e.g., earlier or later, time point. Methods for visually inspecting cell cultures in real time are known in the art. In a specific embodiment, a time period that minimizes variability in cell number is identified if decreased variability in the amount of cells counted or in the percent confluence is detected from cell preparations taken from same cell lot, and/or if decreased variability in the amount of cells counted or in the percent confluence is detected from cell preparations comprising the same cell type but taken from different cell lots is determined.

Studies have demonstrated signals of efficacy in post myocardial infarct patients using MSC, with the idea that the therapeutic effects are mediated by paracrine means [188-191]. Mechanistically, it is believed that MSC produce growth factors that protect cardiomyocytes from death [192-194], stimulate angiogenesis [195-197], inhibit fibrosis [198], reduce infarct size [199], and suppress development of pathologically remodeling [200]. Regenerative immunotherapy utilizes immune cells to produce these same growth factors. In one study, MSC-conditioned medium (CM) was collected from human sources and assessed in a pig model of ischemia and reperfusion injury. The researchers showed that intravenous and intracoronary MSC-CM treatment significantly reduced myocardial nuclear oxidative stress, caspase 3 activation and inhibition of TGF-beta signaling. Furthermore, infarct size was reduced by 60% and augmentation of systolic and diastolic cardiac performance was noted [201]. One of the mechanisms by which MSC-CM may be functioning is through activation/recruitment of cardiac specific stem cells. Numerous groups have demonstrated that endogenous stem cells reside in the heart, and that these cells have ability to help heal injured hearts [202-204]. In a study by Nakanishi et al. MSC-CM promoted proliferation of cardiac stem cells and inhibited their apoptosis. Using transwell chambers it was demonstrated that MSC-CM was chemotactic for cardiac stem cells. Importantly, the MSC-CM increased beta-myosin heavy chain and atrial natriuretic peptide gene expression in the cardiac stem cells, suggesting stimulation of differentiation [205]. In certain embodiments, determination of a time period that minimizes variability in cell number once the cells have been cultured for the time period is accomplished qualitatively. In a specific embodiment, a time period that minimizes variability in cell number is identified if decreased variability in the amount of a surrogate marker, e.g., levels of PGE2, detected from cell preparations taken from same cell lot is determined, and/or if decreased variability in the amount of a surrogate marker, e.g., levels of PGE2, detected from cell preparations comprising the same cell type but taken from different cell lots is determined.

It is known that hypoxia is found in injured tissue. Therefore, in one embodiment of the invention potency of cells used in regenerative immunotherapy, cells such as Treg, their potency is assessed by exposing cells to hypoxia and then assessing growth factor production. It is known that hypoxia induces production of numerous factors such as VEGF [206-212], HGF [213], FGF-2 [214], wnt4 [215], BDNF [216], GDNF [216], exosomes [217, 218], and interleukin-10 [219, 220].

The assays of the invention are to quantify the potency of regenerative immunotherapy for treatment of diseases and disorders. Examples of diseases and disorders include but are not limed to: Cancer which refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Other diseases that are treated by the cells quantified by the current invention are “inflammatory diseases” which refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Other diseases that are treated by the cells quantified by the current invention are “autoimmune diseases”, which refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

Other diseases that are treated by the cells quantified by the current invention are “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

Other diseases that are treated by the cells quantified by the current invention are “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

Other diseases that are treated by the cells quantified by the current invention are “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

Other diseases that are treated by the cells quantified by the current invention are “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

Other diseases that are treated by the cells quantified by the current invention are “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

Other diseases that are treated by the cells quantified by the current invention are “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

Other diseases that are treated by the cells quantified by the current invention are “psychiatric disorder” refers to a disease of the mind and includes diseases and disorders listed in the Diagnostic and Statistical Manual of Mental Disorders-Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, Cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

Other diseases that are treated by the cells quantified by the current invention. The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

### REFERENCES

ADDIN EN.REFLIST 1. Kernie, S. G. and J. M. Parent, Forebrain neurogenesis after focal Ischemic and traumatic brain injury. Neurobiol Dis, 2010. 37(2): p. 267-74.
2. Ton, S. T., et al., Dentate Gyrus Proliferative Responses After Traumatic Brain Injury and Binge Alcohol in Adult Rats. Neurosci Insights, 2020. 15: p. 2633105520968904.
3. Zhou, C., et al., Pentraxin 3 contributes to neurogenesis after traumatic brain injury in mice. Neural Regen Res, 2020. 15(12): p. 2318-2326.
4. Falnikar, A., et al., Differential Response in Novel Stem Cell Niches of the Brain after Cervical Spinal Cord Injury and Traumatic Brain Injury. J Neurotrauma, 2018. 35(18): p. 2195-2207.
5. Chang, E. H., et al., Traumatic Brain Injury Activation of the Adult Subventricular Zone Neurogenic Niche. Front Neurosci, 2016. 10: p. 332.
6. Clausi, M. G., E. Kumari, and S. W. Levison, Unmasking the responses of the stem cells and progenitors in the subventricular zone after neonatal and pediatric brain injuries. Neural Regen Res, 2016. 11(1): p. 45-8.
7. Sun, D., Endogenous neurogenic cell response in the mature mammalian brain following traumatic injury. Exp Neurol, 2016. 275 Pt 3: p. 405-410.
8. Goodus, M. T., et al., Neural stem cells in the immature, but not the mature, subventricular zone respond robustly to traumatic brain injury. Dev Neurosci, 2015. 37(1): p. 29-42.
9. Dixon, K. J., et al., Endogenous neural stem/progenitor cells stabilize the cortical microenvironment after traumatic brain injury. J Neurotrauma, 2015. 32(11): p. 753-64.
10. Thau-Zuchman, O., et al., Combination of vascular endothelial and fibroblastgrowth factor 2 for induction of neurogenesis and angiogenesis after traumatic brain injury. J Mol Neurosci, 2012. 47(1): p. 166-72.
11. Ahmed, A. I., et al., Endogenous GFAP-positive neural stem/progenitor cells in the postnatal mouse cortex are activated following traumatic brain injury. J Neurotrauma, 2012. 29(5): p. 828-42.
12. Chen, X. H., et al., Neurogenesis and glial proliferation persist for at least one year in the subventricular zone following brain trauma in rats. J Neurotrauma, 2003. 20(7): p. 623-31.
13. Dillen, Y., et al., Adult Neurogenesis in the Subventricular Zone and Its Regulation After Ischemic Stroke: Implications for Therapeutic Approaches. Transl Stroke Res, 2020. 11(1): p. 60-79.
14. Zhang, R. L., Z. G. Zhang, and M. Chopp, Ischemic stroke and neurogenesis in the subventricular zone. Neuropharmacology, 2008. 55(3): p. 345-52.
15. Williamson, M. R., T. A. Jones, and M. R. Drew, Functions of subventricular zone neural precursor cells in stroke recovery. Behav Brain Res, 2019. 376: p. 112209.
16. Marques, B. L., et al., The role of neurogenesis in neurorepair after ischemic stroke. Semin Cell Dev Biol, 2019. 95: p. 98-110.
17. Jurkowski, M. P., et al., Beyond the Hippocampus and the SVZ: Adult Neurogenesis Throughout the Brain. Front Cell Neurosci, 2020. 14: p. 576444.
18. Stepien, T., et al., Neurogenesis in adult human brain after hemorrhage and ischemic stroke. Folia Neuropathol, 2018. 56(4): p. 293-300.
19. Taupin, P., Stroke-induced neurogenesis: physiopathology and mechanisms. Curr Neurovasc Res, 2006. 3(1): p. 67-72.
20. Liu, X. S., et al., Stroke induces gene profile changes associated with neurogenesis and angiogenesis in adult subventricular zone progenitor cells. J Cereb Blood Flow Metab, 2007. 27(3): p. 564-74.
21. Liang, H., et al., Region-specific and activity-dependent regulation of SVZ neurogenesis and recovery after stroke. Proc Natl Acad Sci U S A, 2019. 116(27): p. 13621-13630.
22. Ohab, J. J., et al., A neurovascular niche for neurogenesis after stroke. J Neurosci, 2006. 26(50): p. 13007-16.
23. Kuge, A., et al., Temporal profile of neurogenesis in the subventricular zone, dentate gyrus and cerebral cortex following transient focal cerebral ischemia. Neurol Res, 2009. 31(9): p. 969-76.
24. Belayev, L., et al., Docosanoids Promote Neurogenesis and Angiogenesis, Blood-Brain Barrier Integrity, Penumbra Protection, and Neurobehavioral Recovery After Experimental Ischemic Stroke. Mol Neurobiol, 2018. 55(8): p. 7090-7106.
25. Gabriel-Salazar, M., et al., Angiogenin in the Neurogenic Subventricular Zone After Stroke. Front Neurol, 2021. 12: p. 662235.
26. Shang, J., et al., Strong neurogenesis, angiogenesis, synaptogenesis, and antifibrosis of hepatocyte growth factor in rats brain after transient middle cerebral artery occlusion. J Neurosci Res, 2011. 89(1): p. 86-95.
27. Schabitz, W. R., et al., Intravenous brain-derived neurotrophic factor enhances poststroke sensorimotor recovery and stimulates neurogenesis. Stroke, 2007. 38(7): p. 2165-72.
28. Jordan, J., et al., Bone morphogenetic proteins: neurotrophic roles for midbrain dopaminergic neurons and implications of astroglial cells. Eur J Neurosci, 1997. 9(8): p. 1699-709.
29. Luo, Y., et al., Delayed treatment with a p53 inhibitor enhances recovery in stroke brain. Ann Neurol, 2009. 65(5): p. 520-30.
30. Gonzalez, F. F., et al., Erythropoietin increases neurogenesis and oligodendrogliosis of subventricular zone precursor cells after neonatal stroke. Stroke, 2013. 44(3): p. 753-8.
31. Zheng, J., et al., Post-stroke estradiol treatment enhances neurogenesis in the subventricular zone of rats after permanent focal cerebral ischemia. Neuroscience, 2013. 231: p. 82-90.
32. Liu, Y., et al., Progranulin promotes functional recovery and neurogenesis in the subventricular zone of adult mice after cerebral ischemia. Brain Res, 2021. 1757: p. 147312.
33. Plane, J. M., et al., Retinoic acid and environmental enrichment alter subventricular zone and striatal neurogenesis after stroke. Exp Neurol, 2008. 214(1): p. 125-34.
34. Tan, X. D., et al., Gadd45b mediates environmental enrichment-induced neurogenesis in the SVZ of rats following ischemia stroke via BDNF. Neurosci Lett, 2021. 745: p. 135616.
35. Belayev, L., et al., A novel neurotrophic therapeutic strategy for experimental stroke. Brain Res, 2009. 1280: p. 117-23.
36. Matsumori, A., et al., Increased circulating hepatocyte growth factor in the early stage of acute myocardial infarction. Biochem Biophys Res Commun, 1996. 221(2): p. 391-5.
37. Rappolee, D .A., A. Iyer, and Y. Patel, Hepatocyte growth factor and its receptor are expressed in cardiac myocytes during early cardiogenesis. Circ Res, 1996. 78(6): p. 1028-36.
38. Sato, T., et al., Hepatocyte growth factor(HGF): a new biochemical marker for acute myocardial infarction. Heart Vessels, 1997. 12(5): p. 241-6.
39. Ueda, H., et al., Gene transfection of hepatocyte growth factor attenuates reperfusion injury in the heart. Ann Thorac Surg, 1999. 67(6): p. 1726-31.
40. Yasuda, S., et al., Enhanced secretion of cardiac hepatocyte growth factor from an infarct region is associated with less severe ventricular enlargement and improved cardiac function. J Am Coll Cardiol, 2000. 36(1): p. 115-21.
41. Nakamura, T., et al., Myocardial protection from ischemia/reperfusion injury by endogenous and exogenous HGF. J Clin Invest, 2000. 106(12): p. 1511-9.
42. Ueda, H., et al., A potential cardioprotective role of hepatocyte growth factor in myocardial infarction in rats. Cardiovasc Res, 2001. 51(1): p. 41-50.
43. Kitta, K., et al., Hepatocyte growth factor protects cardiac myocytes against oxidative stress-induced apoptosis. Free Radic Biol Med, 2001. 31(7): p. 902-10.
44. Kitta, K., et al., Hepatocyte growth factor induces GATA-4 phosphorylation and cell survival in cardiac muscle cells. J Biol Chem, 2003. 278(7): p. 4705-12.
45. Jin, H., et al., Early treatment with hepatocyte growth factor improves cardiac function in experimental heart failure induced by myocardial infarction. J Pharmacol Exp Ther, 2003. 304(2): p. 654-60.
46. Ahmet, I., et al., Gene transfer of hepatocyte growth factor improves angiogenesis and function of chronic ischemic myocardium in canine heart. Ann Thorac Surg, 2003. 75(4): p. 1283-7.
47. Li, Y., et al., Postinfarction treatment with an adenoviral vector expressing hepatocyte growth factor relieves chronic left ventricular remodeling and dysfunction in mice. Circulation, 2003. 107(19): p. 2499-506.
48. Jayasankar, V., et al., Gene transfer of hepatocyte growth factor attenuates postinfarction heart failure. Circulation, 2003. 108 Suppl 1: p. 11230-6.
49. Jin, H., et al., The therapeutic potential of hepatocyte growth factor for myocardial infarction and heart failure. Curr Pharm Des, 2004. 10(20): p. 2525-33.
50. Ryugo, M., et al., Myocardial protective effect of human recombinant hepatocyte growth factor for prolonged heart graft preservation in rats. Transplantation, 2004. 78(8): p. 1153-8.
51. Nakamura, T., et al., Hepatocyte growth factor prevents tissue fibrosis, remodeling, and dysfunction in cardiomyopathic hamster hearts. Am J Physiol Heart Circ Physiol, 2005. 288(5): p. H2131-9.
52. Sakaguchi, G., et al., Control-released hepatocyte growth factor prevents the progression of heart failure in stroke prone spontaneously hypertensive rats. Ann Thorac Surg, 2005. 79(5): p. 1627-34.
53. Yumoto, A., et al., Hepatocyte growth factor gene therapy reduces ventricular arrhythmia in animal models of myocardial ischemia. Acta Med Okayama, 2005. 59(3): p. 73-8.
54. Rastogi, S., et al., Myocardial transfection with naked DNA plasmid encoding hepatocyte growth factor prevents the progression of heart failure in dogs. Am J Physiol Heart Circ Physiol, 2011. 300(4): p. H1501-9.
55. Siltanen, A., et al., hHGF overexpression in myoblast sheets enhances their angiogenic potential in rat chronic heart failure. PLoS One, 2011. 6(4): p. e19161.
56. Ellison, G. M., et al., Endogenous cardiac stem cell activation by insulin-like growth factor-1/hepatocyte growth factor intracoronary injection fosters survival and regeneration of the infarcted pig heart. J Am Coll Cardiol, 2011. 58(9): p. 977-86.
57. Hahn, W., et al., Enhanced cardioprotective effects by coexpression of two isoforms of hepatocyte growth factor from naked plasmid DNA in a rat ischemic heart disease model. J Gene Med, 2011. 13(10): p. 549-55.
58. Poppe, A., et al., Hepatocyte growth factor-transfected skeletal myoblasts to limit the development of postinfarction heart failure. Artif Organs, 2012. 36(3): p. 238-46.
59. Jin, Y. N., et al., Long-term effects of hepatocyte growth factor gene therapy in rat myocardial infarct model. Gene Ther, 2012. 19(8): p. 836-43.
60. Madonna, R., et al., Hepatocyte growth factor: molecular biomarker and player in cardioprotection and cardiovascular regeneration. Thromb Haemost, 2012. 107(4): p. 656-61.
61. Okayama, K., et al., Hepatocyte growth factor reduces cardiac fibrosis by inhibiting endothelial-mesenchymal transition. Hypertension, 2012. 59(5): p. 958-65.
62. Wang, S., et al., Effects of hepatocyte growth factor overexpressed bone marrow-derived mesenchymal stem cells on prevention from left ventricular remodelling and functional improvement in infarcted rat hearts. Cell Biochem Funct, 2012. 30(7): p. 574-81.
63. Yi, X., et al., Hepatocyte growth factor regulates the TGF-beta1-induced proliferation, differentiation and secretory function of cardiac fibroblasts. Int J Mol Med, 2014. 34(2): p. 381-90.
64. Gallo, S., et al., HGF/Met Axis in Heart Function and Cardioprotection. Biomedicines, 2014. 2(4): p. 247-262.
65. Zhang, J., et al., Hepatocyte growth factor modification enhances the anti-arrhythmic properties of human bone marrow-derived mesenchymal stem cells. PLoS One, 2014. 9(10): p. e111246.
66. Zhang, G. W., et al., HGF and IGF-1 promote protective effects of allogeneic BMSC transplantation in rabbit model of acute myocardial infarction. Cell Prolif, 2015. 48(6): p. 661-70.
67. He, J., et al., Hypoxic adipose mesenchymal stem cells derived conditioned medium protects myocardial infarct in rat. Eur Rev Med Pharmacol Sci, 2015. 19(22): p. 4397-406.
68. Wang, W., et al., Effects of Adenovirus-Mediated Hepatocyte Growth Factor Gene Therapy on Postinfarct Heart Function: Comparison of Single and Repeated Injections. Hum Gene Ther, 2016. 27(8): p. 643-51.
69. Liu, J., et al., Ad-HGF improves the cardiac remodeling of rat following myocardial infarction by upregulating autophagy and necroptosis and inhibiting apoptosis. Am J Transl Res, 2016. 8(11): p. 4605-4627.
70. Sonnenberg, S. B., et al., Delivery of an engineered HGF fragment in an extracellular matrix-derived hydrogel prevents negative LV remodeling post-myocardial infarction. Biomaterials, 2015. 45: p. 56-63.
71. Rao, K. S., et al., Human epicardial cell-conditioned medium contains HGF/IgG complexes that phosphorylate RYK and protect against vascular injury. Cardiovasc Res, 2015. 107(2): p. 277-86.
72. Komarowska, I., et al., Hepatocyte Growth Factor Receptor c-Met Instructs T Cell Cardiotropism and Promotes T Cell Migration to the Heart via Autocrine Chemokine Release. Immunity, 2015. 42(6): p. 1087-99.
73. Gallo, S., et al., Activation of the MET receptor attenuates doxorubicin-induced cardiotoxicity in vivo and in vitro. Br J Pharmacol, 2020. 177(13): p. 3107-3122.
74. Radik, M., et al., Hepatocyte growth factor plays a particular role in progression of overall cardiac damage in experimental pulmonary hypertension. Int J Med Sci, 2019. 16(6): p. 854-863.
75. Ponder, K. P., et al., Therapeutic neonatal hepatic gene therapy in mucopolysaccharidosis VII dogs. Proc Natl Acad Sci U S A, 2002. 99(20): p. 13102-7.
76. Yoshida, J., [An experimental study on the protective effect of hepatocyte growth factor (HGF) for the primary cultured hepatocytes obtained from iron-loaded rats]. Hokkaido Igaku Zasshi, 1995. 70(1): p. 99-111.
77. Matsuda, Y., et al., Preventive and therapeutic effects in rats of hepatocyte growth factor infusion on liver fibrosis/cirrhosis. Hepatology, 1997. 26(1): p. 81-9.
78. Masunaga, H., et al., Preventive effects of the deleted form of hepatocyte growth factor against various liver injuries. Eur J Pharmacol, 1998. 342(2-3): p. 267-79.
79. Ogura, Y., et al., Hepatocyte growth factor promotes liver regeneration and protein synthesis after hepatectomy in cirrhotic rats. Hepatogastroenterology, 2001. 48(38): p. 545-9.
80. Xue, F., et al., Hepatocyte growth factor gene therapy accelerates regeneration in cirrhotic mouse livers after hepatectomy. Gut, 2003. 52(5): p. 694-700.
81. Oyagi, S., et al., Therapeutic effect of transplanting HGF-treated bone marrow mesenchymal cells into CCl4-injured rats. J Hepatol, 2006. 44(4): p. 742-8.
82. Asano, Y., et al., Hepatocyte growth factor promotes remodeling of murine liver fibrosis, accelerating recruitment of bone marrow-derived cells into the liver. Hepatol Res, 2007. 37(12): p. 1080-94.
83. Suzumura, K., et al., Adeno-associated virus vector-mediated production of hepatocyte growth factor attenuates liver fibrosis in mice. Hepatol Int, 2008. 2(1): p. 80-8.
84. Hu, J. J., et al., Therapeutic effect of transplanting beta(2)m(−)/Thy1(+) bone marrow-derived hepatocyte stem cells transduced with lentiviral-mediated HGF gene into CCl(4)-injured rats. J Gene Med, 2010. 12(3): p. 244-54.
85. Jin, S. Z., et al., Hepatocyte growth factor promotes liver regeneration induced by transfusion of bone marrow mononuclear cells in a murine acute liver failure model. J Hepatobiliary Pancreat Sci, 2011. 18(3): p. 397-405.
86. Seo, K. W., et al., Therapeutic effects of hepatocyte growth factor-overexpressing human umbilical cord blood-derived mesenchymal stem cells on liver fibrosis in rats. Cell Biol Int, 2014. 38(1): p. 106-16.
87. Atta, H., et al., Mutant MMP-9 and HGF gene transfer enhance resolution of CCl4-induced liver fibrosis in rats: role of ASHI and EZH2 methyltransferases repression. PLoS One, 2014. 9(11): p. e112384.
88. Tang, W. P., et al., Basic fibroblast growth factor-treated adipose tissue-derived mesenchymal stem cell infusion to ameliorate liver cirrhosis via paracrine hepatocyte growth factor. J Gastroenterol Hepatol, 2015. 30(6): p. 1065-74.
89. Ogaly, H. A., et al., Hepatocyte Growth Factor Mediates the Antifibrogenic Action of Ocimum bacilicum Essential Oil against CCl4-Induced Liver Fibrosis in Rats. Molecules, 2015. 20(8): p. 13518-35.
90. Zhang, Y., et al., Therapeutic effect of hepatocyte growth factor-overexpressing bone marrow-derived mesenchymal stem cells on CCl4-induced hepatocirrhosis. Cell Death Dis, 2018. 9(12): p. 1186.
91. Yin, F., et al., Effect of Human Umbilical Cord Mesenchymal Stem Cells Transfected with HGF on TGF-beta1/Smad Signaling Pathway in Carbon Tetrachloride-Induced Liver Fibrosis Rats. Stem Cells Dev, 2020. 29(21): p. 1395-1406.
92. Shimamura, M., N. Sato, and R. Morishita, Experimental and clinical application of plasmid DNA in the field of central nervous diseases. Curr Gene Ther, 2011. 11(6): p. 491-500.
93. Tsuzuki, N., et al., Hepatocyte growth factor reduces infarct volume after transient focal cerebral ischemia in rats. Acta Neurochir Suppl, 2000. 76: p. 311-6.
94. Tsuzuki, N., et al., Hepatocyte growth factor reduces the infarct volume afteransient focal cerebral ischemia in rats. Neurol Res, 2001. 23(4): p. 417-24.
95. Hayashi, K., et al., Gene therapy for preventing neuronal death using hepatocyte growth factor: in vivo gene transfer of HGF to subarachnoid space prevents delayed neuronal death in gerbil hippocampal CAI neurons. Gene Ther, 2001. 8(15): p. 1167-73.
96. Yoshimura, S., et al., Gene transfer of hepatocyte growth factor to subarachnoid space in cerebral hypoperfusion model. Hypertension, 2002. 39(5): p. 1028-34.
97. Shimamura, M., et al., Novel therapeutic strategy to treat brain ischemia: overexpression of hepatocyte growth factor gene reduced ischemic injury without cerebral edema in rat model. Circulation, 2004. 109(3): p. 424-31.
98. Nagayama, T., et al., Post-ischemic delayed expression of hepatocyte growth factor and c-Met in mouse brain following focal cerebral ischemia. Brain Res, 2004. 999(2): p. 155-66.
99. Zhao, M. Z., et al., Novel therapeutic strategy for stroke in rats by bone marrow stromal cells and ex vivo HGF gene transfer with HSV-1 vector. J Cereb Blood Flow Metab, 2006. 26(9): p. 1176-88.
100. Shimamura, M., et al., Gene transfer of hepatocyte growth factor gene improves learning and memory in the chronic stage of cerebral infarction. Hypertension, 2006. 47(4): p. 742-51.
101. Trapp, T., et al., Hepatocyte growth factor/c-MET axis-mediated tropism of cord blood-derived unrestricted somatic stem cells for neuronal injury. J Biol Chem, 2008. 283(47): p. 32244-53.
102. Hokari, M., et al., Synergistic effects of granulocyte-colony stimulating factor on bone marrow stromal cell transplantation for mice cerebral infarct. Cytokine, 2009. 46(2): p. 260-6.
103. Shang, J., et al., Antiapoptotic and antiautophagic effects of glial cell line-derived neurotrophic factor and hepatocyte growth factor after transient middle cerebral artery occlusion in rats. J Neurosci Res, 2010. 88(10): p. 2197-206.
104. Choi, Y. J., et al., Enhancing trophic support of mesenchymal stem cells by ex vivo treatment with trophic factors. J Neurol Sci, 2010. 298(1-2): p. 28-34.
105. Doeppner, T. R., et al., Acute hepatocyte growth factor treatment induces long-term neuroprotection and stroke recovery via mechanisms involving neural precursor cell proliferation and differentiation. J Cereb Blood Flow Metab, 2011. 31(5): p. 1251-62.
106. Chen, J., et al., Neuroprotective effect of human placenta-derived cell treatment of stroke in rats. Cell Transplant, 2013. 22(5): p. 871-9.
107. Zhang, P., et al., N-Butylphthalide (NBP) ameliorated cerebral ischemia reperfusion-induced brain injury via HGF-regulated TLR4/NF-kappaB signaling pathway. Biomed Pharmacother, 2016. 83: p. 658-666.
108. Tang, H., et al., Delayed recanalization after MCAO ameliorates ischemic stroke by inhibiting apoptosis via HGF/c-Met/STAT3/Bcl-2 pathway in rats. Exp Neurol, 2020. 330: p. 113359.
109. Igawa, T., et al., Hepatocyte growth factor may function as a renotropic factor for regeneration in rats with acute renal injury. Am J Physiol, 1993. 265(1 Pt 2): p. F61-9.
110. Kawaida, K., et al., Hepatocyte growth factor prevents acute renal failure and accelerates renal regeneration in mice. Proc Natl Acad Sci U S A, 1994. 91(10): p. 4357-61.
111. Matsumoto, K. and T. Nakamura, HGF: its organotrophic role and therapeutic potential. Ciba Found Symp, 1997. 212: p. 198-211; discussion 211-4.
112. Wang, S. and R. Hirschberg, Role of growth factors in acute renal failure. Nephrol Dial Transplant, 1997. 12(8): p. 1560-3.
113. Liu, Y., A. M. Sun, and L. D. Dworkin, Hepatocyte growth factor protects renal epithelial cells from apoptotic cell death. Biochem Biophys Res Commun, 1998. 246(3): p. 821-6.
114. Mizuno, S., et al., Hepatocyte growth factor prevents renal fibrosis and dysfunction in a mouse model of chronic renal disease. J Clin Invest, 1998. 101(9): p. 1827-34.
115. Stracke, S., et al., Differentiating and proliferative effects of HGF in renal proximal tubular cells are mediated via different signalling pathways. Nephrol Dial Transplant, 1998. 13(6): p. 1398-405.
116. Vargas, G. A., A. Hoeflich, and P. M. Jehle, Hepatocyte growth factor in renal failure: promise and reality. Kidney Int, 2000. 57(4): p. 1426-36.
117. Haug, C., et al., Inhibitory effect of epidermal growth factor and hepatocyte growth factor on endothelin-1 release by rabbit proximal tubule cells. J Cardiovasc Pharmacol, 2000. 36(5 Suppl 1): p. S248-51.
118. Laping, N J., Hepatocyte growth factor in renal disease: cause or cure? Cell Mol Life Sci, 1999. 56(5-6): p. 371-7.
119. Mizuno, S., K. Matsumoto, and T. Nakamura, Hepatocyte growth factor suppresses interstitial fibrosis in a mouse model of obstructive nephropathy. Kidney Int, 2001. 59(4): p. 1304-14.
120. Vijayan, A., et al., Hepatocyte growth factor inhibits apoptosis after ischemic renal injury in rats. Am J Kidney Dis, 2001. 38(2): p. 274-8.
121. Yang, J., C. Dai, and Y. Liu, Systemic administration of naked plasmid encoding hepatocyte growth factor ameliorates chronic renal fibrosis in mice. Gene Ther, 2001. 8(19): p. 1470-9.
122. Dai, C., J. Yang, and Y. Liu, Single injection of naked plasmid encoding hepatocyte growth factor prevents cell death and ameliorates acute renal failure in mice. J Am Soc Nephrol, 2002. 13(2): p. 411-422.
123. Yamasaki, N., et al., Hepatocyte growth factor protects functional and histological disorders of HgCl(2)-induced acute renal failure mice. Nephron, 2002. 90(2): p. 195-205.
124. Nagano, T., et al., Ameliorative effect of hepatocyte growth factor on glycerol-induced acute renal failure with acute tubular necrosis. Nephron, 2002. 91(4): p. 730-8.
125. Nagano, T., et al., Pre- or post-treatment with hepatocyte growth factor prevents glycerol-induced acute renal failure. Ren Fail, 2004. 26(1): p. 5-11.
126. Dai, C., et al., Intravenous administration of hepatocyte growth factor gene ameliorates diabetic nephropathy in mice. J Am Soc Nephrol, 2004. 15(10): p. 2637-47
127. Gong, R., et al., Hepatocyte growth factor ameliorates renal interstitial inflammation in rat remnant kidney by modulating tubular expression of macrophage chemoattractant protein-1 and RANTES. J Am Soc Nephrol, 2004. 15(11): p. 2868-81.
128. Esposito, C., et al., Hepatocyte growth factor (HGF) modulates matrix turnover in human glomeruli. Kidney Int, 2005. 67(6): p. 2143-50.
129. Franquesa, M., et al., Direct electrotransfer of hHGF gene into kidney ameliorates ischemic acute renal failure. Gene Ther, 2005. 12(21): p. 1551-8.
130. Herrero-Fresneda, I., et al., HGF gene therapy attenuates renal allograft scarring by preventing the profibrotic inflammatory-induced mechanisms. Kidney Int, 2006. 70(2): p. 265-74.
131. Liu, Y. and J. Yang, Hepatocyte growth factor: new arsenal in the fights against renal fibrosis? Kidney Int, 2006. 70(2): p. 238-40.
132. Mizuno, S., K. Matsumoto, and T. Nakamura, HGF as a renotrophic and anti-fibrotic regulator in chronic renal disease. Front Biosci, 2008. 13: p. 7072-86.
133. Homsi, E., et al., Endogenous hepatocyte growth factor attenuates inflammatory response in glycerol-induced acute kidney injury. Am J Nephrol, 2009. 29(4): p. 283-91.
134. Chen, Y., et al., Hepatocyte growth factor modification promotes the amelioration effects of human umbilical cord mesenchymal stem cells on rat acute kidney injury. Stem Cells Dev, 2011. 20(1): p. 103-13.
135. Zarjou, A., et al., Paracrine effects of mesenchymal stem cells in cisplatin-induced renal injury require heme oxygenase-1. Am J Physiol Renal Physiol, 2011. 300(1): p. F254-62.
136. Wang, H. Y., et al., Hepatocyte growth factor-induced amelioration in chronic renal failure is associated with reduced expression of alpha-smooth muscle actin. Ren Fail, 2012. 34(7): p. 862-70.
137. Zhou, D., et al., Activation of hepatocyte growth factor receptor, c-met, in renal tubules is required for renoprotection after acute kidney injury. Kidney Int, 2013. 84(3): p. 509-20.
138. Du, T., et al., Human Wharton's jelly-derived mesenchymal stromal cells reduce renal fibrosis through induction of native and foreign hepatocyte growth factor synthesis in injured tubular epithelial cells. Stem Cell Res Ther, 2013. 4(3): p. 59.
139. Yamaura, K., et al., Suppression of acute and chronic rejection by hepatocyte growth factor in a murine model of cardiac transplantation: induction of tolerance and prevention of cardiac allograft vasculopathy. Circulation, 2004. 110(12): p. 1650-7.
140. Wang, Y., et al., Hepatocyte growth factor prevents ventricular remodeling and dysfunction in mice via Akt pathway and angiogenesis. J Mol Cell Cardiol, 2004. 37(5): p. 1041-52.
141. Tambara, K., et al., Administration of control-released hepatocyte growth factor enhances the efficacy of skeletal myoblast transplantation in rat infarcted hearts by greatly increasing both quantity and quality of the graft. Circulation, 2005. 112(9 Suppl): p. I129-34.
142. Futamatsu, H., et al., Hepatocyte growth factor ameliorates the progression of experimental autoimmune myocarditis: a potential role for induction of T helper 2 cytokines. Circ Res, 2005. 96(8): p. 823-30.
143. Kamimoto, M., et al., Hepatocyte growth factor prevents multiple organ injuries in endotoxemic mice through a heme oxygenase-1-dependent mechanism. Biochem Biophys Res Commun, 2009. 380(2): p. 333-7.
144. Kaido, T., et al., Continuous hepatocyte growth factor supply prevents lipopolysaccharide-induced liver injury in rats. FEBS Lett, 1997. 411(2-3): p. 378-82.
145. Kosai, K., et al., Hepatocyte growth factor prevents endotoxin-induced lethal hepatic failure in mice. Hepatology, 1999. 30(1): p. 151-9.
146. Birukova, A. A., et al., HGF attenuates thrombin-induced endothelial permeability by Tiam1-mediated activation of the Rac pathway and by Tiaml/Rac-dependent inhibition of the Rho pathway. FASEB J, 2007. 21(11): p. 2776-86.
147. Kamimoto, M., S. Mizuno, and T. Nakamura, Reciprocal regulation of IL-6 and IL-10 balance by HGF via recruitment of heme oxygenase-1 in macrophages for attenuation of liver injury in a mouse model of endotoxemia. Int J Mol Med, 2009. 24(2): p. 161-70.
148. Mizuno, S. and T. Nakamura, Improvement of sepsis by hepatocyte growth factor, an anti-inflammatory regulator: emerging insights and therapeutic potential. Gastroenterol Res Pract, 2012. 2012: p. 909350.
149. Shimizu, K., et al., Hepatocyte growth factor inhibits lipopolysaccharide-induced oxidative stress via epithelial growth factor receptor degradation. Arterioscler Thromb Vasc Biol, 2012. 32(11): p. 2687-93.
150. Meng, F., et al., Asef mediates HGF protective effects against LPS-induced lung injury and endothelial barrier dysfunction. Am J Physiol Lung Cell Mol Physiol, 2015. 308(5): p. L452-63.
151. Peng, F., et al., HGF alleviates septic endothelial injury by inhibiting pyroptosis via the mTOR signalling pathway. Respir Res, 2020. 21(1): p. 215.
152. Mizokami, T., et al., Preferential expansion of human umbilical cord blood-derived CD34-positive cells on major histocompatibility complex-matched amnion-derived mesenchymal stem cells. Haematologica, 2009. 94(5): p. 618-28.
153. Nizzardo, M., et al., Minimally invasive transplantation of iPSC-derived ALDHhiSSCloVLA4+ neural stem cells effectively improves the phenotype of an amyotrophic lateral sclerosis model. Hum Mol Genet, 2014. 23(2): p. 342-54.
154. Du, Y., et al., Adipose-derived stem cells differentiate to keratocytes in vitro. Mol Vis, 2010. 16: p. 2680-9.
155. Lin, S. C., et al., Endogenous retinoic acid regulates cardiac progenitor differentiation. Proc Natl Acad Sci U S A, 2010. 107(20): p. 9234-9.
156. Christ, O., et al., Improved purification of hematopoietic stem cells based on their elevated aldehyde dehydrogenase activity. Haematologica, 2007. 92(9): p. 1165-72.
157. Pearce, D.J. and D. Bonnet, The combined use of Hoechst efflux ability and aldehyde dehydrogenase activity to identify murine and human hematopoietic stem cells. Exp Hematol, 2007. 35(9): p. 1437-46.
158. Corti, S., et al., Identification of a primitive brain-derived neural stem cell population based on aldehyde dehydrogenase activity. Stem Cells, 2006. 24(4): p. 975-85.
159. Du, Y., et al., Multipotent stem cells in human corneal stroma. Stem Cells, 2005. 23(9): p. 1266-75.
160. Schwieterman, W. D., FDA perspective on anti-TNF treatments. Ann Rheum Dis, 1999. 58 Suppl 1: p.190-1.
161. Sandborn, W. J. and S. B. Hanauer, Antitumor necrosis factor therapy for inflammatory bowel disease: a review of agents, pharmacology, clinical results, and safety. Inflamm Bowel Dis, 1999. 5(2): p. 119-33.
162. Nicholson, T. E. and K. W. Renton, Role of cytokines in the lipopolysaccharide-evoked depression of cytochrome P450 in the brain and liver. Biochem Pharmacol, 2001. 62(12): p. 1709-17.
163. Cyranowski, J. M., et al., Depressive symptoms and production of proinflammatory cytokines by peripheral blood mononuclear cells stimulated in vitro. Brain Behav Immun, 2007. 21(2): p. 229-37.
164. Hannestad, J., N. DellaGioia, and M. Bloch, The effect of antidepressant medication treatment on serum levels of inflammatory cytokines: a meta-analysis. Neuropsychopharmacology, 2011. 36(12): p. 2452-9.
165. Pasquale, M. D., et al., Early inflammatory response correlates with the severity of injury. Crit Care Med, 1996. 24(7): p. 1238-42.
166. Gerstenfeld, L. C., et al., Impaired fracture healing in the absence of TNF-alpha signaling: the role of TNF-alpha in endochondral cartilage resorption. J Bone Miner Res, 2003. 18(9): p. 1584-92.
167. Lehmann, W., et al., Tumor necrosis factor alpha (TNF-alpha) coordinately regulates the expression of specific matrix metalloproteinases (MIPS) and angiogenic factors during fracture healing. Bone, 2005. 36(2): p. 300-10.
168. Marano, M. A., et al., Cachectin/TNF production in experimental burns and Pseudomonas infection. Arch Surg, 1988. 123(11): p. 1383-8.
169. Marano, M. A., et al., Serum cachectin/tumor necrosis factor in critically ill patients with burns correlates with infection and mortality. Surg Gynecol Obstet, 1990. 170(1): p. 32-8.
170. Endo, S., et al.,Plasma tumour necrosis factor-alpha (TNF-alpha) levels in patients with burns. Burns, 1993. 19(2): p. 124-7.
171. Maury, C. P. and A. M. Teppo, Circulating tumour necrosis factor-alpha (cachectin) in myocardial infarction. J Intern Med, 1989. 225(5): p. 333-6.
172. Lissoni, P., et al., Enhanced secretion of tumour necrosis factor in patients with myocardial infarction. Eur J Med, 1992. 1(5): p. 277-80.
173. Ter Horst, G. J., TNF-alpha-induced selective cerebral endothelial leakage and increased mortality risk in postmyocardial infarction depression. Am J Physiol, 1998. 275(5): p. H1910-1.
174. Wang, Y., et al., TGF-alpha increases human mesenchymal stem cell-secreted VEGF by MEK- and PI3-K- but not JNK- or ERK-dependent mechanisms. Am J Physiol Regul Integr Comp Physiol, 2008. 295(4): p. R1115-23.
175. Zhang, A., et al., Mechanism of TNF-alpha-induced migration and hepatocyte growth factor production in human mesenchymal stem cells. J Cell Biochem, 2010. 111(2): p. 469-75.
176. Wang, M., et al., Human progenitor cells from bone marrow or adipose tissue produce VEGF, HGF, and IGF-I in response to TNF by a p38 MAPK-dependent mechanism. Am J Physiol Regul Integr Comp Physiol, 2006. 291(4): p. R880-4.
177. Crisostomo, P. R., et al., Human mesenchymal stem cells stimulated by TNF-alpha, LPS, or hypoxia produce growth factors by an NF kappa B- but not MK-dependent mechanism. Am J Physiol Cell Physiol, 2008. 294(3): p. C675-82.
178. English, K., et al., IFN-gamma and TNF-alpha differentially regulate immunomodulation by murine mesenchymal stem cells. Immunol Lett, 2007. 110(2): p. 91-100.
179. Kwon, Y. W., et al., Tumor necrosis factor-alpha-activated mesenchymal stem cells promote endothelial progenitor cell homing and angiogenesis. Biochim Biophys Acta, 2013. 1832(12): p. 2136-44.
180. Boyt, D. T., et al., Dose and duration of interferon gamma pre-licensing interact with donor characteristics to influence the expression and function of indoleamine-2, 3-dioxygenase in mesenchymal stromal cells. J R Soc Interface, 2020. 17(167): p. 20190815.
181. Kim, D. S., et al., Enhanced Immunosuppressive Properties of Human Mesenchymal Stem Cells Primed by Interferon-gamma. EBioMedicine, 2018. 28: p. 261-273.
182. Tobin, M. K., et al., Activated Mesenchymal Stem Cells Induce Recovery Following Stroke Via Regulation of Inflammation and Oligodendrogenesis. J Am Heart Assoc, 2020. 9(7): p. e013583.
183. Bulati, M., et al., The Immunomodulatory Properties of the Human Amnion-Derived Mesenchymal Stromal/Stem Cells Are Induced by INF-gamma Produced by Activated Lymphomonocytes and Are Mediated by Cell-To-Cell Contact and Soluble Factors. Front Immunol, 2020. 11: p. 54.
184. Varkouhi, A. K., et al., Extracellular Vesicles from Interferon-gamma-primed Human Umbilical Cord Mesenchymal Stromal Cells Reduce Escherichia coli-induced Acute Lung Injury in Rats. Anesthesiology, 2019. 130(5): p. 778-790.
185. Serejo, T. R. T., et al., Assessment of the Immunosuppressive Potential of INF-gamma Licensed Adipose Mesenchymal Stem Cells, Their Secretome and Extracellular Vesicles. Cells, 2019. 8(1).
186. Boland, L., et al., IFN-gamma and TNF-alpha Pre-licensing Protects Mesenchymal Stromal Cells from the Pro-inflammatory Effects of Palmitate. Mol Ther, 2018. 26(3): p. 860-873.
187. Yi, J. Z., et al., Interferon-gamma suppresses the proliferation and migration of human placenta-derived mesenchmal stromal cells and enhances their ability to induce the generation of CD4(+)CXCR5(+)Foxp3(+)Treg subset. Cell Immunol, 2018. 326: p. 42-51.
188. Kim, S. H., et al., Improvement in Left Ventricular Function with Intracoronary Mesenchymal Stem Cell Therapy in a Patient with Anterior Wall ST-Segment Elevation Myocardial Infarction. Cardiovasc Drugs Ther, 2018. 32(4): p. 329-338.
189. Wang, Z., et al., Rational transplant timing and dose of mesenchymal stromal cells in patients with acute myocardial infarction: a meta-analysis of randomized controlled trials. Stem Cell Res Ther, 2017. 8(1): p. 21.
190. Singh, A., A. Singh, and D. Sen, Mesenchymal stem cells in cardiac regeneration: a detailed progress report of the last 6 years (2010-2015). Stem Cell Res Ther, 2016. 7(1): p. 82.
191. Gao, L. R., et al., Intracoronary infusion of Wharton's jelly-derived mesenchymal stem cells in acute myocardial infarction: double-blind, randomized controlled trial. BMC Med, 2015. 13: p. 162.
192. Eguchi, S., et al., Cardiomyocytes capture stem cell-derived, anti-apoptotic microRNA-214 via clathrin-mediated endocytosis in acute myocardial infarction. J Biol Chem, 2019. 294(31): p. 11665-11674.
193. Jin, H., P. R. Sanberg, and R. J. Henning, Human umbilical cord blood mononuclear cell-conditioned media inhibits hypoxic-induced apoptosis in human coronary artery endothelial cells and cardiac myocytes by activation of the survival protein Akt. Cell Transplant, 2013. 22(9): p. 1637-50.
194. He, A., et al., The antiapoptotic effect of mesenchymal stem cell transplantation on ischemic myocardium is enhanced by anoxic preconditioning. Can J Cardiol, 2009. 25(6): p. 353-8.
195. Santos Nascimento, D., et al., Human umbilical cord tissue-derived mesenchymal stromal cells attenuate remodeling after myocardial infarction by proangiogenic, antiapoptotic, and endogenous cell-activation mechanisms. Stem Cell Res Ther, 2014. 5(1): p. 5.
196. Tang, J., et al., Combination of chemokine and angiogenic factor genes and mesenchymal stem cells could enhance angiogenesis and improve cardiac function after acute myocardial infarction in rats. Mol Cell Biochem, 2010. 339(1-2): p. 107-18.
197. Otto Beitnes, J., et al., Intramyocardial injections of human mesenchymal stem cells following acute myocardial infarction modulate scar formation and improve left ventricular function. Cell Transplant, 2012. 21(8): p. 1697-709.
198. Mias, C., et al., Mesenchymal stem cells promote matrix metalloproteinase secretion by cardiac fibroblasts and reduce cardiac ventricular fibrosis after myocardial infarction. Stem Cells, 2009. 27(11): p. 2734-43.
199. Gandia, C., et al., Human dental pulp stem cells improve left ventricular function, induce angiogenesis, and reduce infarct size in rats with acute myocardial infarction. Stem Cells, 2008. 26(3): p. 638-45.
200. Goto, T., et al., High-mobility group box I fragment suppresses adverse post-infarction remodeling by recruiting PDGFRalpha-positive bone marrow cells. PLoS One, 2020. 15(4): p. e0230392.
201. Timmers, L., et al., Reduction of myocardial infarct size by human mesenchymal stem cell conditioned medium. Stem Cell Res, 2007. 1(2): p. 129-37.
202. Fathi, E., et al., An overview of the myocardial regeneration potential of cardiac c-Kit(+) progenitor cells via PI3K and MAPK signaling pathways. Future Cardiol, 2020. 16(3): p. 199-209.
203. Scalise, M., et al., Heterogeneity of Adult Cardiac Stem Cells. Adv Exp Med Biol, 2019. 1169: p. 141-178.
204. Marunouchi, T., et al., Transplantation of cardiac Sca-1-positive cells rather than c-Kit-positive cells preserves mitochondrial oxygen consumption of the viable myocardium following myocardial infarction in rats. J Pharmacol Sci, 2019. 140(3): p. 236-241.
205. Nakanishi, C., et al., Activation of cardiac progenitor cells through paracrine effects of mesenchymal stem cells. Biochem Biophys Res Commun, 2008. 374(1): p. 11-6.
206. Mayer, H., et al., Vascular endothelial growth factor (VEGF A) expression in human mesenchymal stem cells: autocrine and paracrine role on osteoblastic and endothelial differentiation. J Cell Biochem, 2005. 95(4): p. 827-39.
207. Potier, E., et al., Hypoxia affects mesenchymal stromal cell osteogenic differentiation and angiogenic factor expression. Bone, 2007. 40(4): p. 1078-87.
208. An, S. S., et al., Neuroprotective effect of combined hypoxia-induced VEGF and bone marrow-derived mesenchymal stem cell treatment. Childs Nery Syst, 2010. 26(3): p. 323-31.
209. Jun, E. K., et al., Hypoxic conditioned medium from human amniotic fluid-derived mesenchymal stem cells accelerates skin wound healing through TGF-beta/SMAD2 and PI3K/Akt pathways. Int J Mol Sci, 2014. 15(1): p. 605-28.
210. Page, P., et al., Effect of serum and oxygen concentration on gene expression and secretion of paracrine factors by mesenchymal stem cells. Int J Cell Biol, 2014. 2014: p. 601063.
211. Paquet, J., et al., Oxygen Tension Regulates Human Mesenchymal Stem Cell Paracrine Functions. Stem Cells Transl Med, 2015. 4(7): p. 809-21.
212. Bader, A. M., et al., Hypoxic Preconditioning Increases Survival and Pro-Angiogenic Capacity of Human Cord Blood Mesenchymal Stromal Cells In Vitro. PLoS One, 2015 10(9): p. e0138477.
213. Chang, C. P., et al., Hypoxic preconditioning enhances the therapeutic potential of the secretome from cultured human mesenchymal stem cells in experimental traumatic brain injury. Clin Sci (Lond), 2013. 124(3): p. 165-76.
214. Shearier, E., et al., Physiologically Low Oxygen Enhances Biomolecule Production and Stemness of Mesenchymal Stem Cell Spheroids. Tissue Eng Part C Methods, 2016. 22(4): p. 360-9.
215. Leroux, L., et al., Hypoxia preconditioned mesenchymal stem cells improve vascular and skeletal muscle fiber regeneration after ischemia through a Wnt4-dependent pathway. Mol Ther, 2010. 18(8): p. 1545-52.
216. Wei, L., et al., Transplantation of hypoxia preconditioned bone marrow mesenchymal stem cells enhances angiogenesis and neurogenesis after cerebral ischemia in rats. Neurobiol Dis, 2012. 46(3): p. 635-45.
217. Zhang, H. C., et al., Microvesicles derived from human umbilical cord mesenchymal stem cells stimulated by hypoxia promote angiogenesis both in vitro and in vivo. Stem Cells Dev, 2012. 21(18): p. 3289-97.
218. Salomon, C., et al., Exosomal signaling during hypoxia mediates microvascular endothelial cell migration and vasculogenesis. PLoS One, 2013. 8(7): p. e68451.
219. Jiang, C. M., et al., Effects of hypoxia on the immunomodulatory properties of human gingiva-derived mesenchymal stem cells. J Dent Res, 2015. 94(1): p. 69-77.
220. Xu, L., et al., Hypoxia-induced secretion of IL-10 from adipose-derived mesenchymal stem cell promotes growth and cancer stem cell properties of Burkitt lymphoma. Tumour Biol, 2016. 37(6): p. 7835-42.

1. A method of quantifying potency of an immunologically based regenerative medicine therapy in which said potency is assessed by one or more of the following methods: a) quantification of basal growth factor production; b) quantification of induced growth factor production; c) quantification of basal immune regulatory factor production; d) quantification of induced growth factor production; e) assessment of ability of basal conditioned media from said immunologically based regenerative medicine therapy to reduce apoptosis in cells representing cells of the target tissue to be treated; f) assessment of ability of basal conditioned media from said immunologically based regenerative medicine therapy to induce regeneration in cells representing cells of the target tissue to be treated; g) assessment of ability of induced conditioned media from said immunologically based regenerative medicine therapy to reduce apoptosis in cells representing cells of the target tissue to be treated; and h) assessment of ability of induced conditioned media from said immunologically based regenerative medicine therapy to induce regeneration in cells representing cells of the target tissue to be treated.

2. The method of claim 1, wherein said immunologically based immunotherapy comprises an immune cell that has been reprogrammed by a regenerative cell.

3. The method of claim 2, wherein said reprogramming endows said immune cell with ability to possess regenerative properties.

4. The method of claim 3, wherein said regenerative properties are angiogenic in nature.

5. The method of claim 3, wherein said regenerative properties are neurogenic in nature.

6. The method of claim 3, wherein said regenerative properties are antiapoptotic in nature.

7. The method of claim 3, wherein said regenerative properties are mitogenic in nature.

8. The method of claim 3, wherein said regenerative properties are anti-inflammatory in nature.

9. The method of claim 4. wherein potency of cells is assessed by quantification of ability to stimulate angiogenesis in an in vitro assay.

10. The method of claim 9, wherein said in vitro assay is stimulation of migration of human umbilical vein endothelial cells.

11. The method of claim 9, wherein said in vitro assay is assessment of angiogenic cytokines produced by cells being assayed.

12. The method of claim 11, wherein said angiogenic cytokines are selected from a group comprising of: G-CS, GM-CSF, 1-309, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, FGF-1, FGF-2, FGF-4, FGF-7, GDF-15, GDNF, Growth Hormone, HB-EGF, HGF, IGFBP-1, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-I) 6Ckine, Ax1, BTC, CCL28, CTACK, CXCL16, ENA-78, Tie-2, TPO, TRAIL R4, TREM-1, VEGF-C, VEGFR1 Adiponectin, Adipsin, AFP, ANGPTL4, B2M, BCAM, CA125, CA15-3, CEA, CRP, ErbB2, Follistatin, FSH, GRO alpha, beta HCG, sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Sigiec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, and Galectin-3.

13. The method of claim 11, wherein said cytokines produced by said cells being assayed are produced after stimulation of said cells.

14. The method of claim 13, wherein said cells are stimulated by one or more means selected from a group comprising of: a) hypoxia; b) hypertonic stress; c) hypotonic stress; d) hyperthermia; e) stimulation with a mitogen; f) stimulation with a cytokine; and g) stimulation with a toll like receptor agonist.

15. The method of claim 5, wherein neurogenic potential is assessed by ability to stimulate proliferation of neuronal progenitors in vitro.

16. The method of claim 15, wherein said neuronal progenitors express Mushashi.

17. The method of claim 15, wherein said neuronal progenitors express interferon gamma receptor.

18. The method of claim 1, wherein HGF-1 is used as a method of assessing potency of said regenerative immunotherapy population.

19. The method of claim 18, wherein HGF-1 assessed is assessed subsequent to culture of said regenerative immunotherapy population with anti-CD3 and anti-CD28 antibodies bound to beads.

20. The method of claim 18, wherein regenerative immunotherapy cells are released for use if production of HGF-1 is above 30 nanograms per 1 million regenerative immunotherapy cells stimulated with 10 million DynaBeads for a period of 24 hours.
